(19) United States
(12) Patent Application Publication
(10) Pub. No.: US 2007/0280898 A1
(43) Pub. Date: Dec. 6, 2007

(54) Dermatological compositions

(75) Inventor: Sheri L. Riddle, Marco Island, FL (US)

Correspondence Address: BROOKS, CAMERON & HUEBSCH , PLLC, 1221 NICOLLET AVENUE , SUITE 500, MINNEAPOLIS, MN, 55403, US

(73) Assignee: P & H Enterprises of Marco Inc.

(21) Appl. No.: 11/447,326

(22) Filed: Jun. 6, 2006

(51) Int. Cl. A61K 8/97; A61K 8/37; A61K 36/82; A61K 36/896; A61K 36/87

(52) U.S. Cl. 424/74; 424/753; 424/776; 424/769; 424/729; 424/777; 424/766; 424/70.31

(57) ABSTRACT

Embodiments of the present disclosure include a dermatological composition for the treatment of skin blemishes, more particularly for the treatment of scars and/or stretch marks. The dermatological composition can include 0.1-1.0 parts by weight of bulbus allium (onion) powder, 0.5-5.0 parts by weight of the obroma cacao (cocoa) seed butter, 0.1-2.0 parts by weight of butyrospermum parkii (shea butter), 0.1-3.0 parts by weight of tocopherol acetate, 0.01-0.05 parts by weight of allantoin, 1.0-5.0 parts by weight of punica granatum (pomegranate) seed oil, 1.0-5.0 parts by weight of persea gratissima (avocado) oil, 1.0-5.0 parts by weight of canibis sativa (hemp) seek oil, 0.1-0.25 parts by weight of a preservative, dermatologically-acceptable aqueous carrier 70-75 parts by weight, fragrance optional, for a total of 100 parts by weight, in the form of a cream. Also a method embodiment of improving the appearance of a skin blemish includes the step of applying to the skin blemish a dermatologically-effective amount thereof.

### FIELD OF THE DISCLOSURE

The present disclosure relates to a dermatological composition for use in the improvement of the appearance of scars, as a lotion, and as a foot peel.

### BACKGROUND

People are increasingly seeking to have a more youthful, less wrinkled appearance by using cosmetic compositions containing active agents capable of combating ageing. In addition, people are seeking dermatological compositions that contain natural ingredients capable of treating dry skin, stretch marks, scars, and age spots.

These disorders can be treated in a number of ways including oral ingestion of drugs, injection of drugs, dietary modification, topical applications and other therapeutic methods. Of these treatment methods, the one most preferable and convenient to patients is generally a topical application.

### DETAILED DESCRIPTION

The compounds of the present disclosure can be formulated into suitable therapeutic, dermatological, medical, and/or cosmetic compositions depending on the particular use for which it is to be used. For example, various compositions can have applications as cosmetics, therapeutics, or both.

Embodiments of the present disclosure comprise a combination of natural oils, such as vitis vinifera (grape) seed oil, persea gratissima (avocado) oil, punica granatum (pomegranate) seed oil, tea tree oils, and/or peppermint oils along with other ingredients. These compositions can moisturize the skin, increase skin elasticity, and/or promote healthy skin cells. Some embodiments also improve the appearance of skin blemishes, including scars and/or stretch marks.

Some embodiments of the present disclosure comprise a cream, having a pleasant fragrance depending on whether or not a fragrance is desired in the product, which comprises bulbus allium (onion) powder, granatum (pomegranate) seed oil, canibis sativa (hemp) seed oil, persea gratissima (avocado) oil, tocopherol (vitamin E), butyrospermum parkii (shea butter), theobroma cacao (cocoa) seed butter, a preservative such as diazolidinyl urea and/or iodopropynyl butylcarbamate, de-ionized water or other dermatologically-acceptable aqueous diluent or carrier, and fragrance to the extent desired. Use of other materials, or ingredients, may also be included.

When applied to skin blemishes, such as scars and/or stretch marks, such embodiments improve the appearance of skin blemishes by making them appear softer and smoother. As used herein, “skin blemishes” include defects in the skin such as scars and/or stretch marks. Scars and stretch marks develop as a result of the disruption of the integrity of the dermis, or the deeper layer of skin. The appearance of scars and stretch marks is due to bands of collagen which the body lays to form strong bonds in order to hold the skin together where it was once cut, as seen in surgery or trauma. Stretch marks are similar to scars in that there is a pulling of the skin leading to small linear scars; however, there is no actual separation of the skin as there is with true scar formation. Stretch marks can appear after the skin is stretched excessively. For example, stretch marks can appear during pregnancy, after a growth spurt during puberty, and/or as a result of rapid weight gain. Ideally, a skin blemish is smooth, flat, and blends in with surrounding skin, but sometimes very visible and unattractive skin blemishes occur. In some embodiments, a dermatological composition improves the appearance of such skin blemishes by making them blend more completely in with the surrounding skin, reducing erythema or redness, reducing their induration or hardness, and in subjectively comforting the patient as objective symptoms decrease. Although nothing can make skin blemishes such as scars and/or stretch marks totally disappear, or hurry the natural healing process, such embodiments of the dermatological composition is effective in improving the appearance of scars and/or stretch marks.

Examples of embodiments of the present disclosure are given to illustrate an embodiment of the composition and method of the present disclosure, but are not to be construed as limiting.

### EXAMPLE 1

In one embodiment, a dermatological composition or formulation is prepared from the following ingredients in the following proportions:

Theobroma cacao (cocoa) seed butter 0.5 to 5.0 parts by weight

Butyrospermum parkii (shea butter) 0.1 to 2.0 parts by weight

Bulbus allium (onion) powder 0.1 to 1.0 parts by weight

Punica granatum (pomegranate) seed oil 1.0 to 5.0 parts by weight

Ascorbic acid (vitamin C) 0.1 to 0.5 parts by weight

Tocopherol acetate 0.1 to 3.0 parts by weight

Allantoin 0.01 to 0.05 parts by weight

Sodium EDTA 0.01 to 1.0 parts by weight

Emulsifying wax NF 1.0 to 5.0 parts by weight

Stearic acid 1.0 to 5.0 parts by weight

Capric/caprillic triglycerides 1.0 to 5.0 parts by weight

Glyceryl mono stearate 0.5 to 5.0 parts by weight

Vitis vinifera (grape) seed oil 1.0 to 5.0 parts by weight

Camellia sinensis (Japanese green tea) leaf extract 0.5 to 5.0 parts by weight

Persea gratissima (avocado) oil 1.0 to 5.0 parts by weight

Cannabis sativa (hemp) seed oil 1.0 to 5.0 parts by weight

Limnanthes alba (meadowfoam) seed oil 1.0 to 5.0 parts by weight

Tetradibutyl pentaerithrity hydroxhydrocinnamate 0.10 to 0.25 parts by weight

Triethyanolamine 0.01 to 1.0 parts by weight

Glyceryl mono stearate 0.1 to 1.0 parts by weight

Cetyl alcohol 0.1 to 1.0 parts by weight

Tetradibutyl pentaerithrity hydroxhydroxinnamate 0.10 to 0.25 parts by weight

Preservative such as diazolidinyl urea and/or iodopropynyl butyl carbamate 0.10 to 0.25 parts by weight

Actinidia deliciosa (kiwi) fragrance 0.10 to 0.20 by weight

Mangifera (mango) fragrance 0.10 to 0.25 parts by weight

Cannabis sativa (hemp) and Persea gratissima (avocado) fragrance 0.10 to 0.15 parts by weight

De-ionized water or other aqueous dermatological carrier 70 to 75 parts by weight

For a total of one hundred (100) weight units. The cream formed by the admixture of the foregoing ingredients together can be packaged for use in the improvement of skin blemishes such as scars and/or stretch marks by application thereto.

### EXAMPLE 2

The following specific amount of ingredients were admixed together with the ranges of ingredients listed in Example 1 to form a dermatological composition in a cream form:

Tetradibutyl pentaerithrity hydroxhydrocinnamate 0.14 parts by weight

Allantoin 0.02 parts by weight

Bulbus allium (onion) powder 0.50 parts by weight

Diazolidinyl urea 0.20 parts by weight

Actinidia deliciosa (kiwi) fragrance 0.16 parts by weight

Mangifera (mango) fragrance 0.20 parts by weight

Cannabis sativa (hemp) and Persea gratissima (avocado) fragrance 0.12 parts by weight

De-ionized water or other aqueous dermatological carrier 72 parts by weight

Embodiments of the present disclosure can also include a lotion including grape seed oil, avocado oil, shea butter, aloe barbadensis (aloe) leaf juice, vitamin E, pomegranate seed oil, a preservative such as diazolidinyl urea and iodopropynyl buytl carbamate, de-ionized water or other dermatologically-acceptable aqueous diluent or carrier, and fragrance to the extent desired. Use of other materials/ingredients can also be included. In this embodiment, the lotion can be applied to over-exercised skin, helping to relieve and condition the skin and muscles. For example, pomegranate oil can balance the pH level in the skin and can help to increase skin elasticity. In addition, in some embodiments, the lotion can relieve and condition muscles after strenuous exercise by including vitamin E and avocado oil and applying the compositional embodiment to the skin, or a portion of the skin.

### EXAMPLE 3

In one embodiment, a dermatological composition or formulation is prepared from the following ingredients in the following proportions:

Punica granatum (pomegranate) seed oil 1.0 to 5.0 parts by weight

Vitis vinifera (grape) seed oil 1.0 to 5.0 parts by weight

Emulsifying wax NF 1.0 to 5.0 parts by weight

Persea gratissima (avocado) oil 1.0 to 5.0 parts by weight

Simmondsia chineensis (jojoba) seed oil 1.0 to 5.0 parts by weight

Capric/caprillic triglycerides 1.0 to 5.0 parts by weight

Vegetable glycerine 0.5 to 5.0 parts by weight

Butyrospermum parkii (shea butter) 0.1 to 2.0 parts by weight

Aloe barbadensis (aloe) leafjuice 0.1 to 2.0 parts by weight

Vegetable stearic acid 1.0 to 5.0 parts by weight

Retinal palmitate (vitamin A) 0.1 to 0.5 parts by weight

Ascorbic acid (vitamin C) 0.1 to 0.5 parts by weight

Tocopherol (vitamin E) 0.1 to 0.5 parts by weight

Ergocalciferol (vitamin D) 0.1 to 0.5 parts by weight

Silk amino acids 0.1 to 2.0 parts by weight

Glyceryl mono stearate 1.0 to 5.0 parts by weight

Preservative such as diazolidinyl urea and/or iodopropynyl butyl carbamate 0.1 to 0.25 parts by weight

De-ionized water or other aqueous dermatological carrier 75 to 80 parts by weight

For a total of one hundred (100) weight units. The lotion formed by the admixture of the foregoing ingredients together can be packaged for use in the improvement of the appearance of a portion of the skin.

### EXAMPLE 4

The following specific amount of ingredients were admixed together with the ranges of ingredients listed in Example 3 to form a dermatological composition in a lotion form:

Diazolidinyl urea and iodopropynyl butyl carbamate 0.2 parts by weight

De-ionized water or other aqueous dermatological carrier 79 parts by weight

Embodiments of the present disclosure can further include a formulation for use on the skin on a foot. In one embodiment, the formulation can be in the form of a foot peel, where the foot peel includes tea tree oil, peppermint oil, and de-ionized water. Use of other materials, or ingredients, can also be included. In this embodiment, the foot peel can condition the skin and seal in the natural moisture of the skin when it is applied to the skin on a foot after the skin has been exfoliated. In addition, in some embodiments, the foot peel can be peeled or rolled off of the skin on the foot when the foot peel is dried.

In some embodiments, the foot peel composition can be applied to the skin on the foot to give an invigorating sensation to the skin on the foot. For example, peppermint oil can help circulation and promote new cell growth, causing hard, dry skin to dissipate and be replaced by new, softer skin. In addition, peppermint oil can cool off the skin on the foot, which can relieve painful foot problems or sore feet for hours.

In addition, in some embodiments, the foot peel composition can include tea tree oils. In these embodiments, the tea tree oil can deodorize and prevent fungus from growing on the skin on the foot.

### EXAMPLE 5

In one embodiment, a dermatological composition or formulation is prepared from the following ingredients in the following proportions:

Peppermint oil 0.1 to 2.0 parts by weight

Tea tree oil 0.1 to 2.0 parts by weight

Persea gratissima (avocado) oil 0.5 to 5.0 parts by weight

Simmondsia chineensis (jojoba) seed oil 0.5 to 5.0 parts by weight

Salicylic acid 0.1 to 0.5 parts by weight

Polyacrylamide C13-14 isoparaffin, laureth −71.0 to 5.0 parts by weight

Citric Acid 0.1 to 0.5 parts by weight

Polyvinyl alcohol 5.0 to 10.0 parts by weight

Glyceryl monostearate 1.0 to 5.0 parts by weight

Disodium EDTA 0.05 to 0.25 parts by weight

Preservative such as diazolidinyl urea and/or iodopropynyl butyl carbamate 0.001 to 0.2 parts by weight

De-ionized water or other aqueous dermatological carrier 80 to 85 parts by weight

For a total of one hundred (100) weight units. The foot peel formed by the admixture of the foregoing ingredients together can be packaged for use in the improvement of the appearance of the skin on the foot.

### EXAMPLE 6

The following specific amount of ingredients were admixed together with the ranges of ingredients listed in Example 5 to form a dermatological composition in a foot peel form:

Salicylic acid 0.3 parts by weight

Citric Acid 0.2 parts by weight

Disodium EDTA 0.1 parts by weight

Diazolidinyl urea 0.2 parts by weight

De-ionized water or other aqueous dermatological carrier 83 parts by weight

It is to be understood that the present invention is not to be limited to the exact details of operation, or to the exact compounds, compositions, methods, procedures, or embodiments shown and described, as various modifications and equivalents will be apparent to one skilled in the art, wherefore the present invention is to be limited only by the full scope which can be legally accorded to the appended claims. Although the individual components of the various composition embodiments may have been used previously in dermatological compositions, they have not previously been combined in the manner described herein, particularly in the present proportions, and least of all in the absence of a drug serving as an active ingredient, for the purposes described herein.

In addition, in the foregoing Detailed Description, various features are grouped together in a single embodiment for the purpose of streamlining the disclosure. This method of disclosure is not to be interpreted as reflecting an intention that the embodiments of the disclosure require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed:

1. A dermatological composition comprising:
a formulation for use in improving appearance of a skin blemish of a group consisting of: a scar and a stretch mark, comprising:
0.1-1.0 parts by weight of bulbus allium (onion) powder, 0.01-0.05 parts by weight of allantoin, 0.5-5.0 parts by weight of theobroma cacao (cocoa) seed butter, 0.1-2.0 parts by weight of butyrospermum parkii (shea butter), 1.0-5.0 parts by weight of punica granatum (pomegranate) seed oil, 0.1-3.0 parts by weight tocopherol acetate, 1.0-5.0 parts by weight of persea gratissima (avocado) oil, 1.0-5.0 parts by weight canibis sativa (hemp) seed oil; and
0.10-0.25 parts by weight of a preservative;
where the components are carried by a dermatologically-acceptable aqueous carrier, for a total of 100 parts by weight, in the form of a cream.

2. The dermatological composition of claim 1, further comprising:
0.10-0.25 parts by weight of tetradibutyl pentaerithrity hydroxhydrocinnamate, 0.1-0.5 parts by weight of ascorbic acid (vitamin C), 0.01-1.0 parts by weight of sodium EDTA, 0.01-1.0 parts by weight of triethanolamine, 1.0-5.0 parts by weight of stearic acid, 1.0-5.0 parts by weight of emulsifying wax NF, 0.5-5.0 parts by weight of glyceryl mono stearate, 1.0-5.0 parts by weight of vitis vinifera (grape) seed oil, 1.0-5.0 parts by weight of limnanthes alba (meadowfoam) seed oil, 1.0-5.0 parts by weight of capric/caprillic triglycerides, 0.1-1.0 parts by weight of cetyl alcohol, 0.5-5.0 parts by weight of camellia sinensis (Japanese Green Tea) leaf extract.

3. The dermatological composition of claim 2, comprising the following parts by weight: bulbus allium (onion) powder about 0.50, allantoin about 0.02, diazolidinyl urea about 0.20, tetradibutyl pentaerithrity hydroxhydrocinnamate about 0.14.

4. The dermatological composition of claim 3, wherein the dermatologically-acceptable aqueous carrier includes de-ionized water (H₂O), and the composition comprises about 72 parts by weight of de-ionized water.

5. The dermatological composition of claim 1, comprising the following parts by weight: actinidia deliciosa (kiwi) fragrance about 0.16, mangifera (mango) fragrance about 0.20, and canibis sativa (hemp) and persea gratissima (avocado) fragrance about 0.12.

6. A method of improving appearance of a skin blemish, of a group consisting of a scar and a stretch mark comprising, applying an amount of a composition of claim 1 to cover the scar or the stretch mark.

7. A method of improving appearance of a skin blemish, of a group consisting of a scar and a stretch mark comprising, applying an amount of a composition of claim 4 to cover the scar or the stretch mark.

8. A method of improving appearance of a skin blemish, of a group consisting of a scar and a stretch mark comprising, applying an amount of a composition of claim 5 to cover the scar or the stretch mark.

9. A dermatological composition comprising:
a formulation for use in improving the appearance of a portion of skin, comprising:
1.0-5.0 parts by weight of punica granatum (pomegranate) seed oil, 1.0-5.0 parts by weight of vitis vinifera (grape) seed oil, 1.0-5.0 parts by weight of persea gratissima (avocado) oil, 0.1-2.0 parts by weight of butyrospermum parkii (shea butter), 0.1-2.0 parts by weight of aloe barbadensis (aloe) leaf juice, 0.1-0.5 parts by weight of tocopherol (vitamin E); and
0.1-0.25 parts by weight of a preservative;
where components are carried by a dermatologically-acceptable aqueous carrier, for a total of 100 parts by weight, in the form of a lotion.

10. The dermatological composition of claim 9, further comprising:
1.0-5.0 parts by weight of emulsifying wax NF, 1.0-5.0 parts by weight of simmondsia chinensis (jojoba) seed oil, 1.0-5.0 parts by weight of capric/caprillic triglicerides, 0.5-5.0 parts by weight of vegetable glycerine, 1.0-5.0 parts by weight of vegetable stearic acid, 0.1-0.5 parts by weight of retinal palmitate (vitamin A), 0.1-0.5 parts by weight of ascorbic acid (vitamin C), 0.1-0.5 parts by weight of ergocalciferol (vitamin D), 0.1-2.0 parts by weight of silk amino acids, 1.0-5.0 parts by weight of glyceryl monostearate.

11. The dermatological composition of claim 10, wherein the dermatologically-acceptable aqueous carrier includes de-ionized water (H₂O), and the composition comprises about 79 parts by weight of de-ionized water.

12. A method of improving appearance of skin comprising, applying an amount of a composition of claim 9 to cover the portion of skin.

13. A method of improving appearance of skin comprising, applying an amount of a composition of claim 10 to cover the portion of skin.

14. A method of improving appearance of skin comprising, applying an amount of a composition of claim 11 to cover the portion of skin.

15. A dermatological composition comprising:
a formulation for use on skin of a foot, comprising:
0.1-2.0 parts by weight of tea tree oil, 0.1-2.0 parts by weight of peppermint oil, 0.5-5.0 parts by weight of persea gratissima (avocado) oil, 0.5-5.0 parts by weight of simmondsia chinensis (jojoba) seed oil, 1.0-5.0 parts by weight of polyacrylamide C13-14 isoparaffin, laureth-7, 5.0-10.0 parts by weight of polyvinyl alcohol; and
0.001-0.2 parts by weight of a preservative;
where components are carried by a dermatologically-acceptable aqueous carrier, for a total of 100 parts by weight, in the form of a foot peel.

16. The dermatological composition of claim 15, further comprising:
0.1-0.5 parts by weight of salicylic acid, 0.1-0.5 parts by weight of citric acid, 0.05-0.25 parts by weight of disodium EDTA, 1.0-5.0 parts by weight of glyceryl monostearate.

17. The dermatological composition of claim 16, comprising the following parts by weight: disodium EDTA about 0.1, citric acid about 0.2, salicylic acid about 0.3.

18. The dermatological composition of claim 17, wherein the dermatologically-acceptable aqueous carrier includes de-ionized water (H₂O), and the composition comprises about 83 parts by weight of de-ionized water.

19. A method of improving appearance of skin on the foot comprising, applying an amount of a composition of claim 15 to cover skin on the foot.

20. A method of improving appearance of skin on the foot comprising, applying an amount of a composition of claim 18 to cover skin on the foot.
